(19) United States
(12) Patent Application Publication
(10) Pub. No.: US 2012/0214224 A1
(43) Pub. Date: Aug. 23, 2012

(54) Flow based clinical analysis

(76) Inventor: Eugene Y. Chan, Boston, MA (US)

(21) Appl. No.: 13/374,683

(22) Filed: Jan. 6, 2012

Related U.S. Application Data
(60) Provisional application No. 61/462,337, filed on Feb. 1, 2011.

(51) Int. Cl. C12M 1/34; G01N 33/50; G01N 27/22; G01N 21/47; G01N 27/06; G01N 27/02; C12M 1/40; G01N 21/64

(52) U.S. Cl. 435/287.2; 435/288.7; 422/68.1; 422/82.08; 422/82.05; 422/82.02; 422/82.01; 435/287.1

(57) ABSTRACT

Values of clinical properties are normally measured by taking a sample from a patient, mixing an aliquot with a reagent, placing the mixture into a selected instrument, and measuring a property. If another property is required, another measurement sequence must be created. This can be efficient on a large scale, for example in a centralized laboratory, but is inefficient on a small scale. It is shown that by using measurement systems based on manipulation of flowing streams, clinical assays can be performed by a hand held device. This flow based system allows complex assays to be performed in remote locations with automated portable instruments that can be flexible enough to conduct a wide variety of assays.

This application claims the benefit of the priority of U.S. provisional application 61/462,337, and of U.S. application Ser. No. 13/374,157, each of which is hereby incorporated by reference in its entirety. This application was supported by NASA under contract numbers NNX08CB51P and NNX09CA44C and by NIH under contract number 1R43HL099092-01.

### FIELD OF THE INVENTION

This invention relates to improved systems of clinical analysis. The systems are based on the flow of samples through general-purpose analytical equipment, wherein a small clinical sample is diluted online and mixed, usually sequentially, with one or more assay-specific testing reagents. Assay identification is built into the reagents, and readout of the assay gives a value of a clinical parameter that can be internally standardized. More than one assay can be conducted in the same aliquot of sample. Changing from one assay to another, or one patient to another, or both, requires only a change of which reagents are mixed with the next sample into the system. Because of its flow based analysis system, the system can be incorporated into a handheld device.

### BACKGROUND OF THE INVENTION

Laboratory-based clinical analysis has become very sophisticated in recent years. A sample can be taken from a patient and automatically analyzed for any of a large number of variables. As a result, an individual physician or small physician group seldom does analyses in the office, but instead sends samples for analysis to specialized practices which can take advantage of the economies of scale using large, complex automated analyzers.

This model is ill-suited for diagnosis in remote locations. In particular, the automated analyzer model is not amenable to being used just to test one person for a particular parameter. However, the vast majority of medical tests are necessarily more complex than dipping a piece of litmus paper into a sample. There is a need for a different type of clinical analysis system—one which has the ability to do sophisticated analysis and calculation in a cost-effective way for single samples. The system preferably is portable, and ideally is hand-held or otherwise packaged in a very simple, easy to use format.

In current practice, the process of analyzing a clinical sample is based on combinations of several common steps. Core steps may include separation of cells from plasma or serum; dilution; mixture with analytical reagents; incubation; reading of a value via optical or other methods; and standardization vs. known concentrations of reference materials.

One route to these objectives is by size reduction of assay materials and systems. Several groups have shown that clinical analysis and similar laboratory procedures can be performed on particles that are greatly reduced in size compared to current clinical assay procedures. For example, White and Gilmanshin, in U.S. Pat. No. 7,595,160, use a nucleic-acid based probe of about 7.5-15 kilobases attached to antibodies, and so having an effective length of 10-20 microns for the DNA sequence. Doyle et al, in U.S. Pat. No. 7,709,544, describe a method of making small objects having different zones, by flowing parallel streams through a channel and polymerizing material contained in said streams. These groups have demonstrated that clinical assays can be miniaturized and placed on nanostrips, and that nanostrips can be read by an optical system, for example by laser excitation of chromophores attached to reagents. Pregibon et al (Science (2007), vol 135 p. 1393-1396) shows one form of such a system, with separate areas on a strip for analysis and identification. Other types of particle-based assays include traditional assays performed on beads such as immunoassays and nucleic-acid based assays. These are commercially available from a wide-range of companies and are well-known in the art.

Examples of in-line separation include devices for real-time blood plasma separation based on filtration or the Zwifach-Fung effect (Yang et al. (Lab on a Chip (2006), vol 6, p. 871-880)) which describes a method of introducing a blood sample into a microfluidic device and then performing continuous blood and plasma separation. In-line filtration also includes methods such as reverse osmosis and other types of conventional size or diffusion based isolation methods. These include methods such as field flow fractionation (Caldwell (Analytical Chemistry (1988), v60, p. 959A-71A)). These methods can be introduced or utilized as part of a workflow.

The technologies utilized by commercial instruments for blood analysis include the Coulter method, flow cytometry, light scattering, spectroscopy, and spectrophotometric approaches. Of these approaches, the first three are utilized in cell counters that can perform a full CBC with differential. These methods are capable of measuring cell count and volume. The other methods are utilized when a full CBC is not required. For instance, Orsense's continuous Hematocrit and Hemoglobin device uses a method called occlusion spectroscopy (McMurdy et al., 2008), which requires temporarily stopping blood flow in a finger while determining Hct and Hb content. The HemoCue systems use a dual wavelength spectrophotometer to determine either Hct/Hb or WBC counts (Gupta et al., 2008; Munoz et al., 2005; Osei-Bimpong et al., 2008). While the detection part of these methods is flow based, none of them can perform a continuous flow-based detection from sample to analysis.

The Coulter principle relies on measuring changes in electrical conductance across a pore of defined size (Arnutti et al., 1997; Coulter, 1956; Kubitschek, 1971). The method is an electrical sensing method. Upon passage of a particle through the aperture, a certain volume of electrolyte is displaced and measured as a voltage change. The size of the voltage change is proportional to the volume of the particle and is given by the following equation: ΔR=ρVₚ/A², where R is the electrical resistance, ρ is the specific resistance of the electrolyte solution, Vₚ the cell volume, and A the area of the aperture.

Light scattering is utilized to determine cell size and morphology (Chu, 2007). Most approaches utilize a laser as the light source. Scattered light is measured at various angles. At 0°, it is called axial light loss, or the difference between light incident on the detector with and without the particles. At other angles, between 0° and 90°, it is called intermediate angle scattering which gives information on cell complexity. The 90° scatter is the side scatter and gives information about the cell's granularity. The light can be polarized or depolarized to give more information. Light scattering approaches that utilize many detectors are called multi-angle light scattering (MALS) methods. Cell-Dyn instruments utilize multiple angles and also polarized light in an approach called multi-angle polarized scatter separation (MAPSS) (Chow and Leung, 1996). Cytochemical staining, such as via Sudan Black or Chlorazol Black (Kass, 1981; Sheenan and Storegy, 1947), are accepted methods to augment the effectiveness of the approach for discriminating between white blood cells. Oxazine 750 is for enhanced detection of reticulocytes in ALL (Kim and Kantor, 1995).

The third approach, fluorescent flow cytometry, utilizes time-of-flight LIF to characterize cells (Shapiro, 2003). Fluorescently stained cells are flowed past a laser using hydrodynamic focusing in flow cytometry. Using a system of dichroic mirrors, bandpass mirrors, and detectors, fluorescence is captured for each cell. Multiple excitation and emission wavelengths increase the amount of information gathered. Fluorescence is captured at different angles to optimize the signal-to-noise. Fluorescent stains can also differentiate between live and dead cells (Lebaron et al., 1998) and nucleic acid content (Tarnok, 2008). At present, none of the prior art methods utilize an integrated continuous flow system from unprocessed sample to detection, analysis, and result. All these methods focus on one component of the workflow and none provide an integrated approach as required as required for a handheld diagnostic device.

Microfluidic flow-focusing in 2-dimensions has yet to be routinely employed for hematocrit (Hct) measurements and cell counting. Conventional flow cytometry utilizes a 3D sheath flow nozzle to generate a coaxial stream of cells within an outer sheath fluid (FIG. 4). 2D geometries perform 1D flow-focusing and 3D geometries can perform 2D flow-focusing. Microfluidic 3D focusing geometries require more involved steps and design than typical planar fabrication (Chung et al., 2003; Howell et al., 2008; Huang et al., 2006; Kummrow et al., 2009; Miyake et al., 1997; Morgan et al., 2003; Morimoto et al., 2009; Sundararajan et al., 2004; Vykoukal et al., 2003; Yang et al., 2004). In a 3D micromachined structure, Neukammer's group was able to demonstrate flow focusing in 2D and applied it to the analysis of various types of blood cells (Kummrow et al., 2009). Cell volume measurements were not determined in this study, as required for hematocrit determination. 2D focusing has been applied to nanoparticles (Morgan et al., 2003), formation of emulsions (Huang et al., 2006; Morimoto et al., 2009), cell sorting (Yang et al., 2004), yeast cell enumeration (Rodriguez-Trujillo et al., 2008), dye characterization (Hairer et al., 2007; Howell et al., 2008; Miyake et al., 1997; Sundararajan et al., 2004), microsphere studies (Chung et al., 2003; Rodriguez-Trujillo et al., 2008; Scott et al., 2008; Simonnet and Groisman, 2005; Vykoukal et al., 2003). In a direct comparison with 2D geometries using microspheres, Chung et al. 2003 found that 3D geometries yielded more uniform intensity signals (Chung et al., 2003).

There are several methods described in the art for mixing microfluidic streams in a flow chamber. These are briefly discussed in Chen and Jang (“Recent Patents on Micromixing Technology”, Recent Patents on Mechanical Engineering 2009, 2, 240-247). A first alternative is a Dean-effect spiral, as described for instance in Ji et al (U.S. Pat. No. 7,160,025) and Sundarsen and Ugaz (Lab Chip 2006 6, 74-82). A second alternative is an expansion step in a stream. (cf Sundarsen and Ugaz, PNAS 103 (19) 7228-7233, 2006).

However, none of these systems has been commercialized at present. One of the major impediments to large scale use appears to be the accurate translation of instrument readings into reliable numbers useful in diagnoses. A particular challenge is the co-processing of data so that the report of the analysis, when issued (electronically or on paper), links together the identity of the assay with the actual test result on the sample, including a reading of the test result against a calibration curve. To meet system objectives, this should be an automated function, not dependent on the skill of the operator of the instrument.

Another option for improving analysis is to minimize and automate the sample handling that needs to be done. Standard automated methods are known, but require complex systems to automate the variety of different procedures that may be requested.

The inventive system has been designed to alleviate the difficulties of existing systems, and in particular to automatically couple both assay identification information and a calibration curve with the data from a patient sample, in a simple to operate hand-held package.

### SUMMARY OF THE INVENTION

In one aspect, the invention comprises a system for obtaining clinical data on individual samples, where the system is capable of being operated in a remote environment by personnel not dedicated to such tasks. In another aspect, the invention comprises a system for calibrating an assay combined with other functions. In another aspect, the invention comprises a system for identification of an assay in an efficient way. In another aspect, the invention comprises a clinical assay system, characterized in using flow-based preparation and analysis to determine values of one or more clinical parameters of a sample. A separation of blood into plasma and blood cells may be performed in a continuous flow separator, wherein blood cells are collected and plasma is discarded or in which blood cells are discarded and plasma is collected. The collected fraction may be further subdivided. A continuous-flow diluter may be used to adjust the concentration of one or more blood components prior to assay, or used to mix a reagent with a flow of a blood component. Dilution may be accomplished by placing a sample for analysis inside a capillary and flowing a fluid over at least a distal tip of said capillary for a predetermined period to obtain a sample with a known dilution. Dilution may be used to provide an appropriate concentration of cells or particles for one-by-one detection.

A sample may be mixed with detection reagents in a continuous flow step. The means of mixing may be selected from one or more of a porous straw, a spiral mixer, and a flow-based chaotic mixer. Dilution may be used to provide an appropriate concentration of cells to allow cell sizing and counting by measuring the longitudinal electric impedance as a cell passes through an orifice in a cannula.

Preferably, all components of the system can be flushed with a cleaning solution, which is preferably collected in a waste-disposal system. Certain properties of the sample are determined by continuous observation of a flow of the sample using one or more detection methods. Detection methods mat be selected from one or more of light absorption, fluorescence, phosphorescence, light scattering, light polarization, electrical resistance, electrical impedance, and variation in electrical capacitance.

Properties of the sample may be determined by the use of analytical particles which are added to the flow steam, or the properties may be determined by the binding of components of the sample to one or more types of analytical particle. Additional reagents may be bound to a component to be assayed, said component being bound to an analytical particle. Additional reagents may carry functional groups that respond to externally-applied detection methods. Preferably, the sample is diluted sufficiently that particles travel through the detection zone approximately one at a time.

The functional groups may be detectable by one or more of light absorption, fluorescence, phosphorescence, light scattering, light polarization, electrical resistance, electrical impedance, and variation in electrical capacitance. A cleaning cycle may be automatically added to any assay performed by the instrument.

The invention encompasses a system for continuous flow-based clinical analysis, wherein the system uses a hand held instrument in which separation and analytical steps are performed, and wherein flow-based sample preparation is used to select, isolate or dilute the component to be analyzed, and wherein flow-based analytical systems are used as required to dilute samples, incorporate analytical reagents, and mix components, and wherein treated samples flow through at least one detector system, which measures at least one property of the sample and sends the results to a data processing system, and wherein after said measurement, samples flow to a waste-collection system. In addition, properties of samples may be measured by the addition of analytical reagents which react with components as part of an assay system. At least one reagent may be particulate in nature. At least one reagent may bind to a component of the sample.

A clinical assay system comprises a handheld, portable instrument that is able to perform a complete analysis using less than 100 microliters of a blood or bodily fluid sample, including the steps of sample preparation, mixing, dilution, reagent delivery, and flow-based detection and analysis. The dilution is preferably sufficient that particles travel through the detection zone approximately one at a time, and preferably the dilution is sufficient that particles travel through the detection zone approximately one at a time.

### DESCRIPTION OF THE DRAWINGS

FIG. 1 compares the analysis system of the invention with that of the current art. The key difference is that the current system typically has several different steps, which are performed in different places or times, while the system of the invention operates as a continuous flow.

FIG. 2 is a diagram of a possible embodiment of the invention in a microfluidic format. In this particular embodiment, several inlets are available for feeding samples and optionally additional reagents into a spiral vortex mixer, which then may be mixed with one or more additional reagents and passed through a detector. This can be accomplished with a single pressure source. In contrast to the prior art, this approach does not have any interrupted steps in the analysis, and integrates more than one critical feature of the diagnostic analysis testing cycle. This workflow is applicable for numerous clinical specimen types, and includes hematology, chemistry and other types of clinical tests.

FIG. 3 shows another embodiment of the invention in which there are two inlets, one for saline and another for a blood sample, which are then micro-mixed and flow focused in a circular channel, prior to analysis and results. In this example, the spiral micromixer mixes the sample. The blood or bodily fluid is directed into port (1) or port (2) and the reagent or saline is directed into the opposite port. The combined sample exits the center of the spiral micromixer at port (7) and is directed towards port (4) for flow focusing via tubing or other microfluidic passageway (6). Saline is directed into port (3) as the sheath flow focusing fluid. The sample is delivered smoothly, without interruption from start to finish, yielding rapid results.

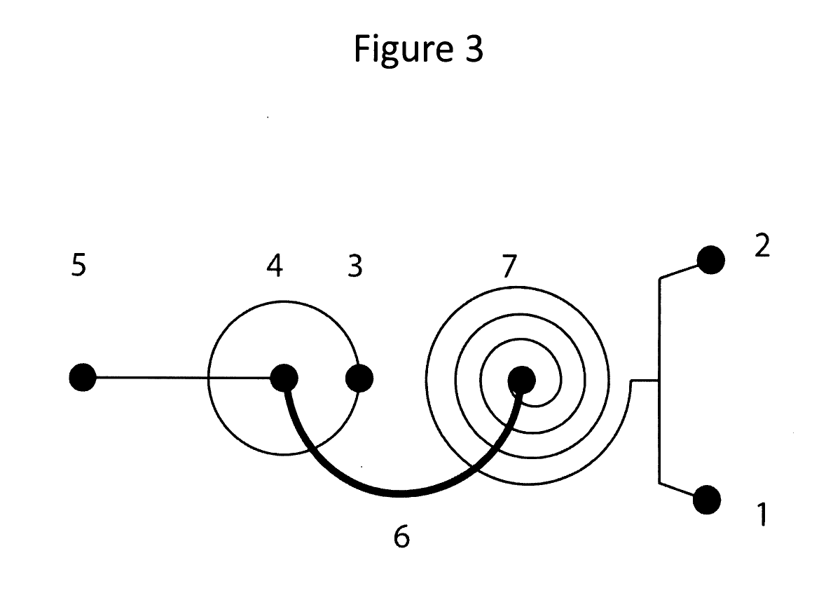

FIG. 4 shows another embodiment of an integrated flow-based analysis method. This approach combines sample introduction, plasma separation, reagent introduction, a micromixer, and a means of detection, all in a continuous flow-based format. The blood sample is introduced into the inlet, then through a cell counting area, then into a Y-junction which separates the blood cells and the plasma. The plasma is directed into a region where reagents are introduced. These reagents are mixed with the plasma in the chaotic mixer and then detected in the downstream detection area using a flow-based detection method. Particle-based assays can be introduced into the reagent inlets for particle analysis and detection in the detection region. Otherwise or in addition, in-solution reagents can also be introduced.

### DETAILED DESCRIPTION OF THE INVENTION

Clinical assay instruments are well developed. With current methods, a hospital or clinic can have a technician draw venous blood into several tubes (typically having different chemistries), which can be placed into systems which will deliver detailed measurements within a few hours, (and for some assays, or if given priority status, within minutes, especially in an emergency. Other body fluids can likewise be collected and placed in an analysis system, such as urine, saliva, mucus and other secretions, and homogenized samples of solid or semi-solid tissue or other substances, such as semen and feces. However, most assay systems of this sort are not well suited for locations that do not have a high throughput of assays. Moreover, automation of the sort presently employed tends toward complex, expensive systems requiring careful maintenance and detailed training of operators.

There is a need for systems that are less complex than large scale analytical systems, yet capable of providing a variety of tests when needed. There is also a need for systems that are simple enough that they can be operated with minimal training. In particular, there is a need to reduce the sample volume required for a complete analysis, so that a complete blood analysis can be obtained with a simple finger prick (less than 100 microliters) rather than a multi-tube venipuncture, and samples of similar size of other tissues and fluids can be used for other assays.

In the following discussion, the term “flow-based system” is used to describe a system that conducts a procedure by a continuous flow of components through at least mixing, processing and analysis steps. A “continuous flow based system” is used for a system that conducts all operations during or after an analysis in a flowing manner. A “blood component” can be any or all of the plasma, or the cells in blood, or a fractionated portion of blood. The term “blood cells” include any cell in the blood, in particular red cells, white cells and platelets, and other cells types such as circulating cancer cells, unless otherwise stated. The term “plasma” includes serum unless otherwise stated. While blood is a major analyte, application to other tissue systems is inferred from references to blood unless it is clear that such an application is inoperable or defective. As examples and without limitation, the system is potentially suitable for analysis of other body liquids, such as sweat, tears, milk, saliva, bile and urine, and for analysis of fluids extracted from other tissues.

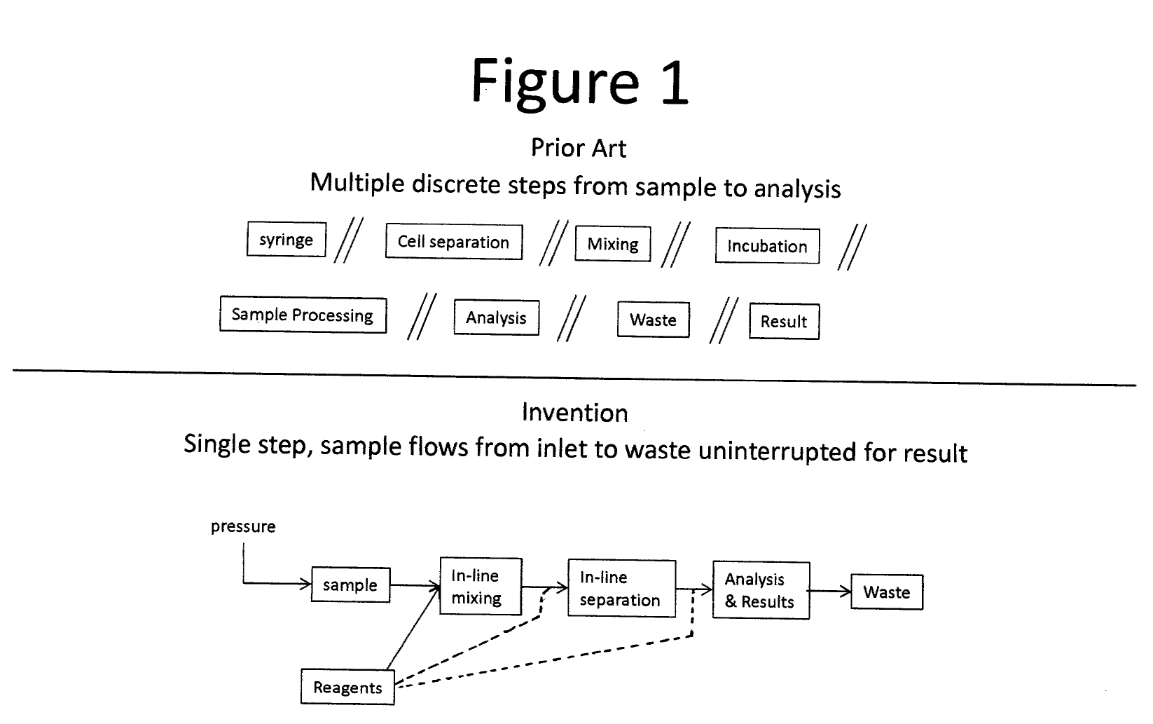

FIG. 1 is a cartoon comparing current analysis systems (upper panel) with the system of the invention (lower panel). In a typical current system), a syringe-full of blood is treated, for example by centrifugation, to separate blood plasma from blood cells. One fraction is selected as the analyte, and that fraction—cells or plasma—is transported to a clinical system for processing. One or more processes will be conducted to obtain a processed analyte which will be ready for quantitation. Next, the processed analyte will be placed in an instrument so that the amount of the material in question can be measured. A result is reported. In most current clinical analysis systems, one or more transfers of material between different devices or between systems is common. In particular, intra-system transfers become more common as the system is less automated.

In the system of the invention, as outlined in the lower panel of FIG. 1, a separation of blood cells from plasma does not require a centrifuge or other isolated separation process. Rather, as seen in the cartoon of FIG. 1, in the system of the invention, the separation of cells from native fluids is done in a flow-based manner. For example, a sample can be passed through a tube with walls that are porous to saline and to plasma, but not to cells. The porous tube may be placed inside a non-porous tube having a suitable clearance with respect to the porous tube. The outer tube may be continuously flushed with saline, so that blood and saline flow in parallel and interchange liquids and small molecules through pores or equivalent. After sufficient passage of time and/or distance, blood cells remain inside the tube and are suspended in saline, while the plasma is outside the tube and has been reduced in concentration in saline to a level that is suitable for automated analysis. Such a reduction may be reproducibly controlled in extent by one or more of pressure drop, flow velocity, and relative volume. If additional dilution of the cells in needed for the assay, cells can be collected from the porous tube and introduced into a flowing stream of saline, and optionally other compounds needed in the intended assay. Alternatively, the proteins and other targets of measurement in the external fluid can be further diluted downstream, if needed. Such variables can be built into a control system and can then be selected by operators if required for a particular assay.

An alternative for a first step is shown in FIG. 1, where the dotted lines show alternative or additional steps of reagent addition. In this example, blood sample is flowed together with a flow of saline in an ILS (in-line separator), where, at the distal end of the separation device, saline becomes the primary suspending medium for the cells, and the dissolved components, such as plasma or serum proteins and other small-sized components, now populate the flowing cell-free side. Either or both of these fractions can be processed further in the same clinical analyzer.

In the simplest systems of the invention, valves or other connectors will be adjusted so that a blood separation results in the collection of either blood cells or plasma (in both cases mixed with saline or other fluid) for further in-line processing. In such cases, a second sample is used if needed to determine values of the other component. In a more complex system, each of blood cells and plasma is separately sent downstream to another separation step if required. Moreover, if the assay does not require separation of blood cells from serum, the separation step may be bypassed.

The flow-based blood separation is in contrast to a conventional separation, in which one of plasma and cells is collected, and the other is discarded, after a centrifugation. This results in either a manual or an automated step in which the other component is removed and the apparatus is rinsed. In the present invention, while there is preferably a system-wide flushing and cleaning step after each separation, it is simply a purging of the system with saline, optionally with cleansing materials, (such as with 10% sodium hypochlorite solution), before flowing the next sample into the system, or after completion of analysis. No manual processes are required, and a simple machine can run many different assays with a simple push of a button.

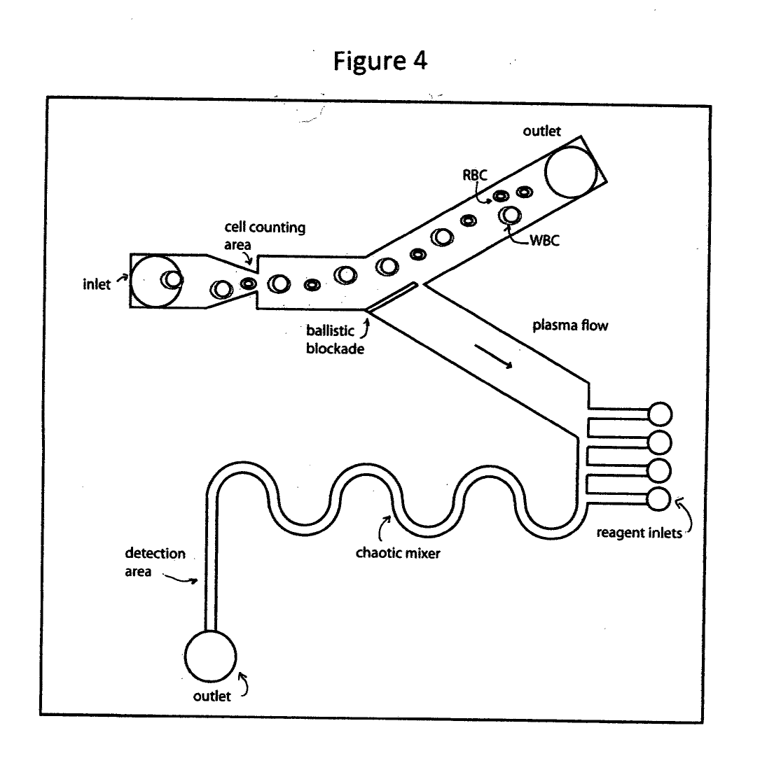

Not all assays require steps beyond simple dilution. For example, counting cells is commonly done, and the key preparation step is to dilute the cells to an appropriate level before counting them, for example by measuring the longitudinal electric impedance as a cell passes through an orifice in a cannula (e.g., a Coulter counter or equivalent). Removal of plasma is generally not necessary. Such assays are also flow-based, and could for example use a capillary pipette in a single-step rinsing setup like that shown in FIG. 4, to introduce cells into an analytical system, which allows control of the level of dilution by controlling the flow rate and the pressure across the capillary.

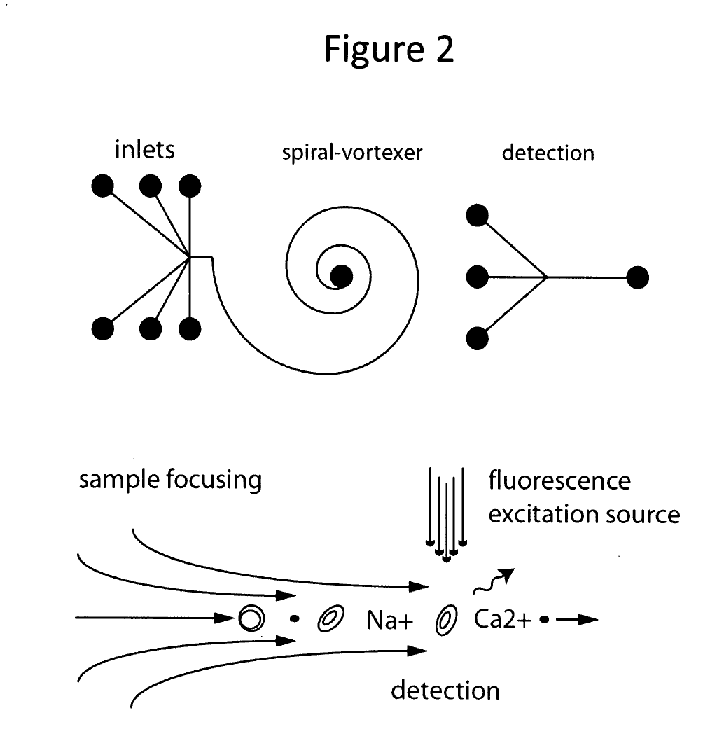

A second important step, with or without prior separation of sample components, is mixing of flowing sample with detection reagents. This can be done at more than one location if required, such as either or both of before and after the in-line mixing example of FIGS. 2 and 4. Detection reagents are materials that interact with components of the sample to allow the components to be detected, and preferably (in most assays) to be quantified. In many assays, a preferred form of detection reagent is a labeled binding molecule that binds to an analyte and thereby attaches a tag specifically to that analyte. Examples of such binding agents include antibodies, substrates or analogs of substrates, and complementary members of multipeptide assemblies, such as complex enzymes, or polynucleotides or polysaccharides. Multiple additions may be made at various stages in the processing, as illustrated in FIG. 2.

Assays of the invention will typically include two different entities that recognize the analyte and bind to it. A first entity will carry a detection reagent, for example a fluorescent tag, to allow detection of bound molecules, and a second entity will carry a group causing binding of a detection reagent to the analyte to facilitate separation of unbound tags, and thus allow quantitation.

Mixing of reagents with sample during labeling, and subsequent separation of unused label from sample, may also be accomplished by in-line continuous flow systems. As an example of the operation of such a system, a sample can be mixed with a detection label by combining a flow containing sample with a flow containing detection label into a single stream for a defined distance (and hence, for a defined mixing time under standard conditions). Then the mixture can be further mixed with a solution containing particles having binding sites for the analyte in the sample. After a further flow incubation, the particles can be separated in-line from other reagents, for example by passage through a porous-walled capillary that prevents passage of the particles through the wall. Such a passage could also be located inside a larger passage through which washing fluid is passed. The washing fluid equilibrates with the fluid inside the capillary and removes molecules not bound to particles. Finally, the cleaned particles flow through a detection cell, and the amount of the analyte is determined by the optical or other properties of the detection label that is complexed to the analyte. All of this is flow-based, i.e., accomplished during a continuing flow and intermixing of fluids containing particles and other materials. Any of the multiple addition pathways for reagents shown in FIG. 1 can be used in such a system, Various separators can be used as well, for example for simple separations of particles and soluble materials, for example as shown in FIG. 4, an example of an in-line separator.

For these purposes, a “particle” can be any object that does not rapidly settle in water, but which is big enough to readily be filtered out of water. Small latex or other particles, for example of 5 micron diameter, can readily be used in the invention. Larger and more complex particles are also suitable, such as micron-scale diagnostic strips containing multiple zones.

Certain combinations of steps have been cited in examples above, but any of a variety of separation and fluid manipulation techniques or steps can be used in the invention. For example, mixing can include any type of mixer, in particular a spiral mixer, a chaotic mixer, a serpentine channel, a herringbone mixer, and similar devices found in the art. Dilution can be in-line dilution, or a serial dilution circuit. Lysing can be accomplished in any convenient way, including mixing lysing reagents (e.g., detergent, protease) with the sample. A preferred method of reagent introduction into the system is via inlet ports that connect to the sample flow stream. Flow focusing can be used both as one dimensional hydrodynamic focusing, and as two dimensional hydrodynamic focusing. Detection and one-by-one analysis of cells and particles can be performed by electrical impedance, laser analysis, light scattering, fluorescence, and other techniques known in the art, and combinations thereof. Flushing can be accomplished by the introduction of saline or other cleaning solution into the system for cleaning. In addition, since everything in the device is based on continuous flow, then the device can be structured so that it can readily be cleaned and used for multiple samples by flow-through cleaning procedures.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference, where such incorporation is permitted. Nothing herein is to be construed as an admission that the invention is not entitled to antedate such disclosure by virtue of prior invention, where relevant. Those skilled in the art will recognize, or be able to ascertain using no more than routine experimentation, many equivalents to the specific embodiments of the invention described herein. Such equivalents are intended to be encompassed by the following claims.

1. A continuous flow-based clinical assay system, characterized in using flow-based preparation and analysis to determine values of one or more clinically-relevant parameters of a sample, and characterized in that at least one of mixing, washing, dilution, separation, labeling and detection of a component is performed using a continuous flow mixer or separator during passage of a sample through the system.

2. The continuous flow-based clinical assay system of claim 1 wherein two or more instances of at least one of mixing, washing, dilution, separation, labeling and detection of a component are combined in a continuous flow mixer or separator during passage of a sample through the system.

3. The continuous flow-based clinical assay system of claim 1 wherein three or more instances of at least two of mixing, washing, dilution, separation, labeling and detection of a component are combined in a continuous flow mixer or separator during passage of a sample through the system.

4. The continuous flow-based clinical assay system of claim 1 wherein the collected fraction is further subdivided.

5. The continuous flow-based clinical assay system of claim 1 wherein a continuous-flow diluter is used to adjust the concentration of one or more blood components prior to assay.

6. The continuous flow-based clinical assay system of claim 1 wherein a continuous-flow diluter is used to mix a reagent with a flow of a blood component.

7. The continuous flow-based clinical assay system of claim 1 wherein a sample is mixed with detection reagents in a continuous flow step.

8. The continuous flow-based clinical assay system of claim 7 wherein the means of mixing are selected from one or more of a porous straw, a spiral mixer, and a flow-based chaotic mixer.

9. The continuous flow-based clinical assay system of claim 1 wherein dilution is used to provide an appropriate concentration of cells or particles for one-by-one detection.

10. The continuous flow-based clinical assay system of claim 1 wherein all components of the system can be flushed with a cleaning solution which is collected in a waste-disposal system.

11. The continuous flow-based clinical assay system of claim 1 wherein properties of the sample are determined by continuous observation of a flow of the sample using one or more detection methods.

12. The continuous flow-based clinical assay system of claim 11 in which the detection methods are selected from one or more of light absorption, fluorescence, phosphorescence, light scattering, light polarization, electrical resistance, electrical impedance, and variation in electrical capacitance.

13. The continuous flow-based clinical assay system of claim 1 in which properties of the sample are determined by the use of analytical particles which are added to the flow steam.

14. The continuous flow-based clinical assay system of claim 1 in which the properties are determined by the binding of components of the sample to one or more types of analytical particle.

15. The continuous flow-based clinical assay system of claim 1 in which additional reagents carry functional groups that respond to externally-applied detection methods.

16. The continuous flow-based clinical assay system of claim 15 in which the functional groups are detectable by one or more of light absorption, fluorescence, phosphorescence, light scattering, light polarization, electrical resistance, electrical impedance, and variation in electrical capacitance.

17. The continuous flow-based clinical assay system of claim 1 which is capable of detection of particles in a flow-focused stream.

18. The continuous flow-based clinical assay system of claim 1 wherein a detection step comprises at least one of flow-focused detection, one-by-one particle detection, and flow cytometry detection.

19. A clinical assay system for continuous flow-based clinical analysis, wherein the system uses a hand held instrument,
wherein flow-based sample preparation is used to select, isolate or dilute the component to be analyzed, and
wherein flow-based analytical systems are used as required to dilute samples, incorporate analytical reagents, and mix components, and
wherein treated samples flow through at least one detector system, which measures at least one property of the sample and sends the results to a data processing system.

20. The clinical assay system of claim 19 wherein a separation of blood into plasma and blood cells is performed in a continuous flow separator.

21. The system of claim 19 wherein properties of samples are measured by the addition of analytical reagents which react with components as part of an assay system.

22. The system of claim 19 wherein at least one reagent is particulate in nature.

23. A clinical assay system, comprising a handheld, portable instrument that is able to perform a complete analysis using less than 100 microliters of a blood or bodily fluid sample, including the steps of flow-based detection and analysis, in addition to one or more of sample preparation, mixing, dilution, and reagent delivery.

24. The clinical assay system of claim 23 in which a dilution step is sufficient that particles travel through the detection zone approximately one at a time.
